(19) United States
(12) Patent Application Publication
(10) Pub. No.: US 2003/0150464 A1
(43) Pub. Date: Aug. 14, 2003

(54) Inducing apoptosis of atrial myocytes to treat atrial fibrillation

(76) Inventor: S. Ward Casscells, Houston, TX (US)

Correspondence Address: C. Steven McDaniel, Esq., McDaniel & Associates, P.C., P.O. Box 2244, Austin, TX, 78768-2244, US

(21) Appl. No.: 09/740,147

(22) Filed: Dec. 18, 2000

Related U.S. Application Data
(60) Provisional application No. 60/172,181, filed on Dec. 17, 1999.

(51) Int. Cl. A61B 18/04

(52) U.S. Cl. 128/898; 606/27; 607/96

(57) ABSTRACT

The present invention relates to methods for treating atrial fibrillation. More specifically, highly active foci of atrial myocytes are identified and selectively heated to induce apoptosis of the arrhythmic foci. The methods generally involve heating the myocytes with a catheter for a sufficient time and at a sufficient temperature to induce programmed cell death.

### CROSS REFERENCE TO PROVISIONAL APPLICATION

This application claims the benefit of U.S. Provisional Application No. 60/172,181 filed Dec. 17, 1999.

### GOVERNMENT INTEREST

The following invention was supported in part through a 1999 Department of Defense Grant DREAMS (Disaster Relief and Emergency Medical Services) grant to the University of Texas Health Sciences Center in Houston, Tex.

### FIELD OF THE INVENTION

The present invention generally relates to methods and devices for the apoptosis by heating alone and by heating in combination with other inducers of apoptosis of special subgroups of atrial myocytes to eliminate ectopic electrical activity, for the prevention of atrial fibrillation.

### BACKGROUND OF THE INVENTION

Atrial fibrillation (AF) is the most common cardiac arrhythmia. It is estimated that 2.2 million Americans have AF, intermittently or permanently (Feinberg et. al. 1995). It is most prevalent in the elderly, with an annual incidence of 1000 person-years of 3.1 cases in men and 1.9 cases in women 55 to 64 years of age, rising to 38.0 and 31.4 cases respectively in the ninth decade of life (Benjamin et. al. 1994). Although most common in those with organic heart disease, it is also encountered in people who consume alcohol, in those with any severe infection, chest injury or after an operation, especially following cardiac or any other thoracic surgery.

AF immediately decreases the cardiac output and blood pressure and can even precipitate shock in patients with certain forms of underlying heart disease. It can cause congestive heart failure in patients with heart disease and, over a period of weeks to months, in those without heart disease if poor rate control results in gradual loss of systolic ventricular function. AF also increases the risk of systemic thromboembolism and is thus a major cause for stroke, with a 2.6 to 4.5-fold risk of stroke after risk factor adjustment (Wolf et. al. 1991 and 1996).

The medications used to treat AF are far from ideal and their efficacy is poor. Although these drugs are more effective than placebo, 50% or more of the patients treated with Class I or Class III anti-arrhythmic drugs have one or more recurrences at the end of one year (Juul-Moller et. al. 1990). Even more importantly, 15 to 30% of the patients treated with these drugs have intolerable side effects requiring termination (Gold et. al. 1986). Proarrhythmia is another serious problem; torsade de pointes, a potentially life-threatening ventricular arrhythmia may occur with an annual risk of 1.5 to 3%, and may be caused by Class I as well as Class III anti-arrhythmic drugs (Prystowsky 1996). Clinically significant bleeding from heparin and warfarin used

[symbol]

[symbol]

[symbol]

it can destabilize a fracture or precipitate hemorrhage and involves an additional risk of stroke in patients who cannot have systemic anticoagulation due to a medical contraindication.

There is a pressing need for new, nonpharmacologic methods of treatment for AF. Recent findings indicate that in many patients with paroxysmal AF, atrial ectopic beats originating from arrhythmogenic left atrial myocytes within or at the ostia (where they meet the left atrium) of the pulmonary veins are the precipitating factors of AF (Haissaguerre et. al. 1998 and Chen et. al. 1999). Several centers have recently reported success in ablating these foci and preventing AF (Haissaguerre et. al. 1998 and Chen el. al. 1999). It is also recognized, however, that mechanical trauma, thrombosis in situ, and especially pulmonary vein stenosis are among the risks of this novel treatment technique. There are reports of hemodynamically significant iatrogenic pulmonary vein scarring and stenosis requiring relief by stent placement or by surgery (Chen et. al. 1999). There is also a risk of proarrhythmia from the scarring and an associated risk of thromboembolism.

Consequently there is an existing need for a non-injurious method of eliminating the arrhythmogenic foci in the pulmonary veins.

### SUMMARY OF THE INVENTION

The present invention provides novel methods and devices for treating AF.

The invention in one regard relates to methods of treating AF or of eliminating an arrhythmogenic focus in a pulmonary vein by inducing apoptosis of an atrial myocyte without causing the collateral damage to the tissue seen in prior art approaches. Carefully controlled heating, in some instances aided by other triggers of apoptosis, is chiefly achieved by ensuring that the heat applied to the atrial myocytes causing the fibrillation is done at a temperature in the range of about 38° C. to 48° C. In certain preferred embodiments, the heating is conducted at a temperature in the range of about 40° C. to 46° C., and in others the heating is conducted at a temperature in the range of about 42° C. to 44° C.

The methods of the invention for treating AF also require that the heating is conducted over a limited time period range, which time period in certain embodiments is one of about 5 to 60 minutes. In certain preferred embodiments, the heating is conducted over a time period range of about 5 to 30 minutes, and in still others the heating is conducted over a time period range of about 5 to 15 minutes.

In the methods of the invention, prior to treating for AF it is preferred that steps are taken to detect the location of and presence of an arrhythmogenic focus, typically comprising group of errant atrial myocytes causing an ectopic misfiring. In certain embodiments, it will be preferred to detect these ectopic electrical discharges using an electrical detector. In others, the location of the site to be treated will be achieved by monitoring the presence of atrial myocytes using positron emission tomography. Where positron emission tomography is used, it will be preferred to track differential uptake of a radio-contrast agent by the atrial myocytes, such as the radio-contrast agent is 18-fluorodeoxyglucose. In other preferred embodiments, the monitoring step will utilize thermogenic detection of groups of atrial myocytes whose collective temperature is elevated over that of the ambient vessel wall temperature. Such detection can be achieved using the devices such as those described in detail in U.S. Pat. No. 5,935,075 (Casscells et al. 1999).

The methods of the invention also preferably include steps for detecting the endpoint of treatment for the AF. Such an endpoint will be at that point where the culprit atrial myocytes are no longer capable of causing the errant electrocardiographic pulses, typically when they are dead. The steps outlined above for detection the presence of the atrial myocytes are equally applicable for detecting their absence.

The methods of the invention may also be practiced using alternative approaches for inducing cell death in the errant atrial myocytes through apoptosis. Alternative triggers to be used in combination with controlled heating may include pharmaceutical approaches such as contacting the atrial myocytes with an effective amount of tumor necrosis factor alpha. Other triggers of apoptosis may be mechanical in nature, such as applying an effective amount of surface pressure to the atrial myocytes, or causing effective amounts of stretching of the atrial myocytes. Such additional triggers may also include metabolic approaches such as causing hypoxia or hypoglycemia in the atrial myocytes. Toxicants may be used as triggers in a similar fashion, such as causing acidosis in the atrial myocytes, or by application of oxidants to the atrial myocytes.

Certain preferred methods of the invention for treating AF by inducing apoptosis of an atrial myocyte will require heating at a temperature in the range of about 38° C. to 48° C. over a time period range of about 5 to 60 minutes. This method may be enhanced by first locating the errant cells to be treated. It may also be enhanced by post-heat monitoring for effective apoptosis of the culprit cells. It may also be enhanced by using, in addition to the controlled heating, at least one additional trigger of apoptosis applied to the atrial myocytes. In one regard, the present invention is a substantial improvement over the prior art methods of ablation for treating AF.

The invention also relates to devices for eliminating an arrhythmogenic focus in a pulmonary vein. The devices of the invention have a heating element capable of maintaining a temperature in the range of about 38° C. to 48° C. over a time period range of about 5 to 60 minutes. These devices also have at least one detector capable of detecting the presence or absence of an arrhythmogenic focus comprising atrial myocytes in any of the ways described above. In certain preferred embodiments, the detector is capable of both detecting the presence of atrial myocytes and of monitoring the removal of said atrial myocytes, though there is no prior reason that multiple detectors could not as easily be used. In one such embodiment a feedback system such as a thermistor or infrared-sensing chip or fiber is used to monitor the temperature of the pulmonary vein. This is used to 1) confirm that apoptosis has been achieved, 2) minimize the duration of heating, and 3) avoid thermal injury. The system would signal when the chosen tissue temperature is reached. Moreover, as apoptosis is initiated and the cellular metabolic and mechanical activity begins to decline, the tissue temperature will fall slightly. Detection of this change will signal to the operator that heating can be discontinued. Such feedback can also be programmed to automatically terminate the heating process. This servomechanism can be used as an alternative or adjunct to deciding to terminate heating based on the typical electrophysiological analysis of electrical conduction.

Certain embodiments of the present invention identify highly active foci of atrial myocytes and specifically heat the identified foci to induce the apoptotic process in the overactive myocytes.

One embodiment of the present invention is a device for mapping the electrical activity of the myocytes in the atrium wall. Optional thermistors or thermocouples may be incorporated into the device to allow the concurrent mapping of temperature and electrical activity of the atrium wall.

Another embodiment of the present invention heats highly active foci of atrial myocytes with a catheter device for a sufficient time and at a sufficient temperature to induce apoptosis in the heated myocytes.

### BRIEF DESCRIPTION OF THE DRAWINGS

FIG. 1. Technical Control: photomicrograph of canine lung tissue inmmunostained for apoptosis as described in the section titled Example, below. The brown nuclei exhibit the characteristic DAB reaction product and morphology of apoptosis. (Original magnification ×40).

FIG. 2. Photomicrograph of canine pulmonary vein heated by radio frequency to 65 degrees C. Extensive necrosis is seen with overlying thrombus and early inflammatory response. (H&E, original magnification ×10).

FIG. 3. Photomicrograph of the tissue in FIG. 2 (heated to 65 degrees C.). TUNEL immunostaining shows no apoptotic cells despite considerable background stain (e.g., the brown deposit along the lumen). (Original magnification ×10).

FIG. 4. Photomicrograph of right upper pulmonary vein within 0.2 cm of the left atrial ostium. The medial layer contains a large number of atrial myocytes. After heating to 45 degrees for 20 minutes, no gross histological damage is seen. (H&E stain, original magnification ×10).

FIG. 5. Photomicrograph of the TUNEL immunostain of the same vein as FIG. 4, shows positive endothelial and subendothelial cells, demonstrating that gentle heat can produce apoptosis of atrial myocytes without necrosis, thrombosis, or inflammation. (Original magnification ×40).

FIG. 6. Photomicrograph of right lower pulmonary vein within 0.2 cm of the left atrial ostium. The media contains a large number of atrial myocytes. After heating to 45 degrees for 20 minutes, no gross histological damage is seen. (H&E, original magnification ×10).

FIG. 7. Photomicrograph of TUNEL stain on section of same vein shows positive (brown) endothelial and subendothelial cells. (Original magnification ×40).

FIG. 8. Photomicrograph of left upper pulmonary vein about 0.5 cm from the left atrial ostium. No heating was performed. (Negative control; H and E)

FIG. 9. Photomicrograph of TUNEL immunostain of the same vein as in FIG. 8. No apoptotic cells are seen.

### DETAILED DESCRIPTION OF THE PREFERRED EMBODIMENT

The present invention provides novel methods that can be used to treat AF. Certain disclosed methods are particularly useful for inducing apoptosis in localized atrial myocyte clusters that cause AF.

There is a pressing need for a nonpharmacologic and non-injurious method of eliminating the arrhythmogenic foci in the pulmonary veins. Preferred embodiments of the present invention induce apoptosis (programmed cell death) in the left atrial myocytes investing the pulmonary veins. Apoptosis is a natural phenomenon in which cells that are programmed to die during embryogenesis or which are faced with certain death due to exposure to high levels of oxidants or radiation or deprivation of glucose or oxygen or extremes of temperature, can often manage to “commit suicide” without jeopardizing the rest of the organism (James 1998). This sparing is achieved because the cell that undergoes apoptosis dies in a way that avoids lysis. Bursting of the cell membranes releases microbes (if the cell is infected) and toxic enzymes and oxidants. In contrast, when the cell undergoes apoptosis, it synthesizes new RNA which encodes caspases, enzymes which neatly cleave the cells' DNA and shrink the cell. The cell then expresses antigens which cause neighboring cells to engulf it and thereby recycle the nutrients but not any microbes, which die during the process of apoptosis. Consequently, apoptosis does not trigger thrombosis, inflammation and scarring.

Apoptosis has been well investigated as a tool in cancer therapy. There has been some research on cardiac apoptosis, though none related to the arrhythmia inducing apoptosis field. Currently, ablation is carried out routinely by radio frequency energy (750 kHz) which heats the myocardial tissue to 60-140 degrees Celsius eliminating the arrhythmogenic focus by coagulative necrosis, at the same time causing subendocardial and transmural scarring, endocardial damage and secondary thrombosis (Huang 1998).

There are many ways by which one might try to trigger the apoptotic process in cardiac myocytes, but a relatively simple technique is that of thermal apoptosis. This has been used with some success in oncology because cancer cells are more sensitive to thermal apoptosis than noncancerous cells. It is also known that proliferating cells and other cells with high metabolism are also more susceptible to thermal apoptosis. The electrically overactive atrial myocytes involved in AF are also sensitive to thermal apoptosis.

The identification of the electrically overactive atrial myocyte clusters provide an added degree of selectivity to the treatment—e.g., heat would be directed to the arrhythmogenic foci to minimize damage to normal tissue. Since the arrhythmic foci are more prone to thermal apoptosis, this selectivity provides further protection against damage to normal tissue.

The identification of atrial myocyte clusters of increased activity can be identified by at least several types of procedures. These same procedures, among others, may be used to monitor for the absence of such atrial myocytes, as well.

One device for detecting electrically overactive atrial myocytes is an adaptation of the Cardiac Pathways, Inc. (Sunnyvale Calif.) basket catheter. Numerous electrodes are positioned to spring out the guided catheter when it is deployed, producing a nearly round array of electrodes which fill up the atrium, permitting the electrodes to contact the wall of the atrium. These electrodes detect and measure the electrical activity along the wall of the atrium. The electrical activity of the atrium is mapped and timed. Optional thermistors or thermocouples may be placed next to each electrode to allow the simultaneous mapping of the temperature of the atrium wall.

An alternative means of detecting myocytes of increased activity is the deployment of positron emission tomography, using 18-fluorodeoxyglucose scanning. The hyperactive cardiac muscle cell will take up more glucose than normal muscle cells and would therefore by identifiable by positron emission tomography.

A third method of identifying overactive myocytes uses an infrared ballon catheter system. The balloon would be used to press up against the atrium wall and permit an infrared image to be obtained without interference by blood. Care must be taken that the catheter not fill the whole atrium at one time because that would obstruct blood flow and precipitate shock. Thus, segments of the atrium wall would be mapped in sequence. A preferred embodiment of this method utilizes a catheter equipped with a piezoelectric sensory system or any magnetic system to assist in determining the location of the image being mapped.

One of the hallmarks of apoptosis as opposed to necrosis is DNA fragmentation. The enzyme terminal deoxynucleotidyl transferase (TdT) preferentially labels DNA in apoptotic cells. ApopTag kits (Intergen Company, Purchase, N.Y.) may be used to detect the DNA fragmentation by selectively labeling the 3′-OH termini of the fragments with modified nucleotides (digoxigenin-dNTP) by TdT using standard immnunohistochemistry techniques on formalin-fixed, paraffin-embedded tissue. (Gold 1994).

Once the foci of overactive myocytes has been localized, the arrhythmic foci would be heated for a sufficient time and at a sufficient temperature to induce localized apoptosis of the overactive myocytes. The myocytes can be heated by a catheter containing an electrical resistance means of directly heating the localized myocytes. The localized myocytes can also be heated indirectly by heat induction using external radio frequency or ultrasound.

The overactive myocytes will be heated a sufficient time and temperature to induce apoptosis. The time and temperature of treatment will be balanced such that lower temperature treatments will typically run for a longer time and shorter time treatments will be used for higher temperature treatments. Heating times will be varied from about 5 minutes to about an hour. Temperatures used will range from about 38.5° C. to about 47° C. One embodiment will treat the myocytes for about 10 minutes at about 42° C.

### EXAMPLE

Under a protocol approved by the University of Texas-Houston Medical School's and the Texas Heart Institute's Animal Welfare Committees, four dogs underwent cardiac catheterization under general anesthi [symbol] [symbol] [symbol] vein, the left inferior pulmonary vein, and the right inferior pulmonary vein were subjected to 20 minutes of thermal treatment generated by an ATAKR-II power generator (1-60 watts, 40-300 ohms, Medtronic, Inc., Minneapolis, Minn.). Dogs were heparinized and maintained at an ACT between 250 and 300 seconds. The catheters and sheaths were removed and the animals allowed to recover.

Two hours later, the animals were sacrificed using an excess of general anesthesia. The left atrium with pulmonary veins attached was removed for gross and histological studies to investigate the structural and histological effects of the thermal treatment. Tissues were sectioned and placed in OCT for frozen sectioning, or 10% formalin for routine processing for paraffin sectioning. Samples were also preserved in glutaraldehyde for electron microscopy. Sections were stained in hematoxylin and eosin or inmmunostained using the ApopTag Plus Peroxidase in situ detection kit for apoptosis, using anti-digoxigenin horseradish peroxidase and diaminobenzidine (Intergen, Inc., Purchase, N.Y., www.intergenco.com). Slides were then counter-stained with methyl green pyronine and examined and photographed (Nikon Diaphot).

As shown in the figures, these experiments demonstrated the feasibility of ablating atrial myocytes by using gentle heating to cause apoptosis without causing thrombosis and inflammation.

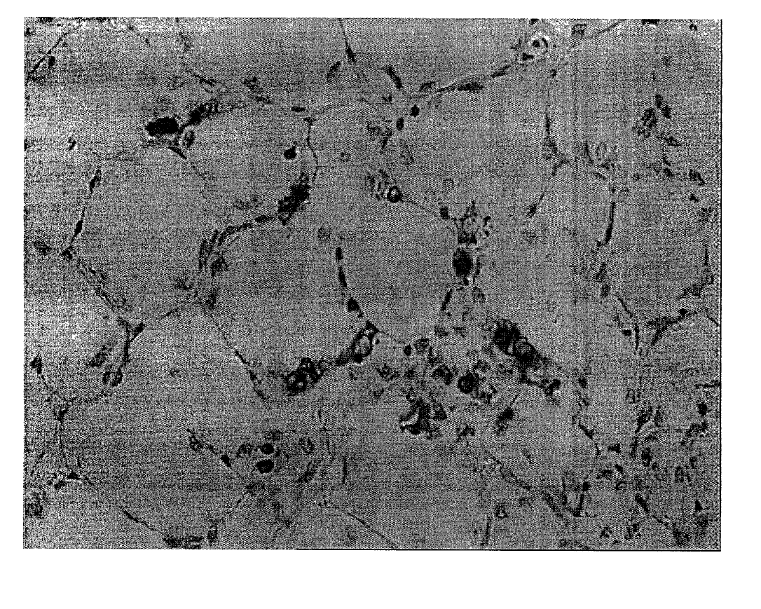

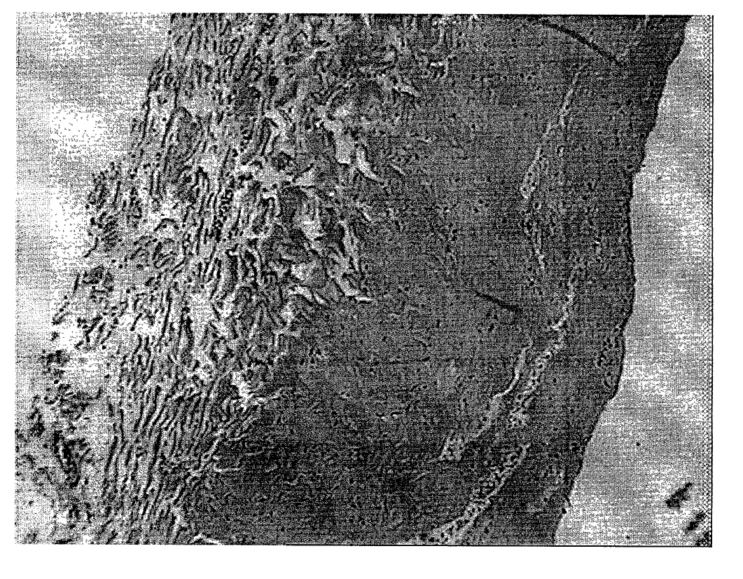

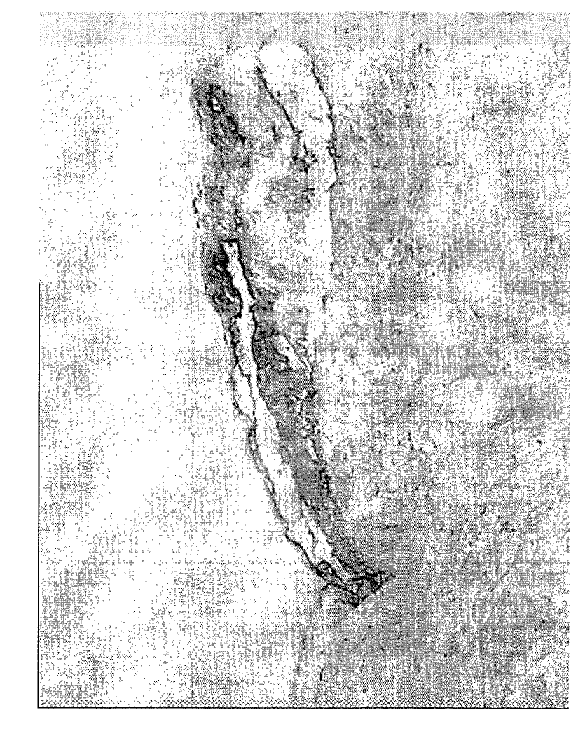

FIG. 1 is a technical control showing a photomicrograph of canine lung tissue inmmunostained for apoptosis as described above. The brown nuclei exhibit the characteristic DAB reaction product and morphology of apoptosis. FIG. 2 is a photomicrograph of a canine pulmonary vein heated by radio frequency to 65 degrees C. using prior art methods of ablation to control AF. It can be seen that there is extensive necrosis with overlying thrombus and early inflammatory response. In FIG. 3, there is shown a photomicrograph of the tissue in FIG. 2 (heated to 65 degrees C. using the prior art approach to ablation to treat AF). TUNEL immunostaining demonstrates that there are no apoptotic cells despite considerable background stain (e.g., the brown deposit along the lumen). Thus, such intense heating, though it may eliminate atrial myocytes, does not do so by inducing apoptosis, and it induces considerable collateral damage to the tissue.

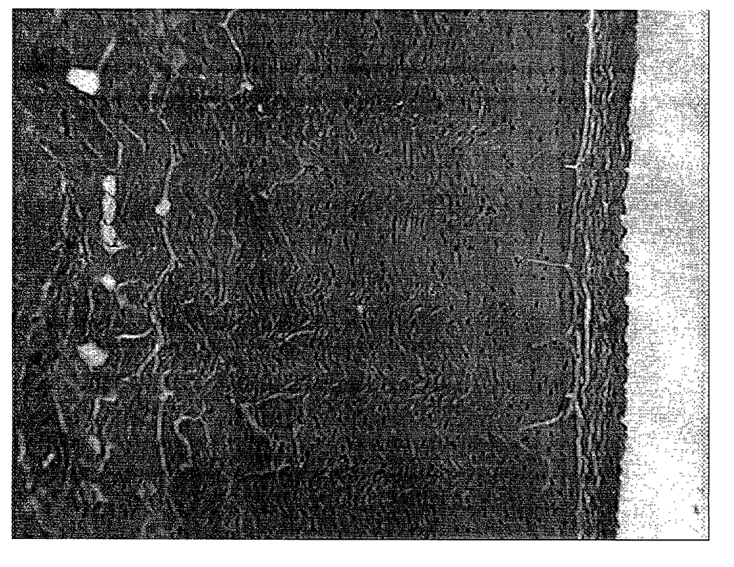

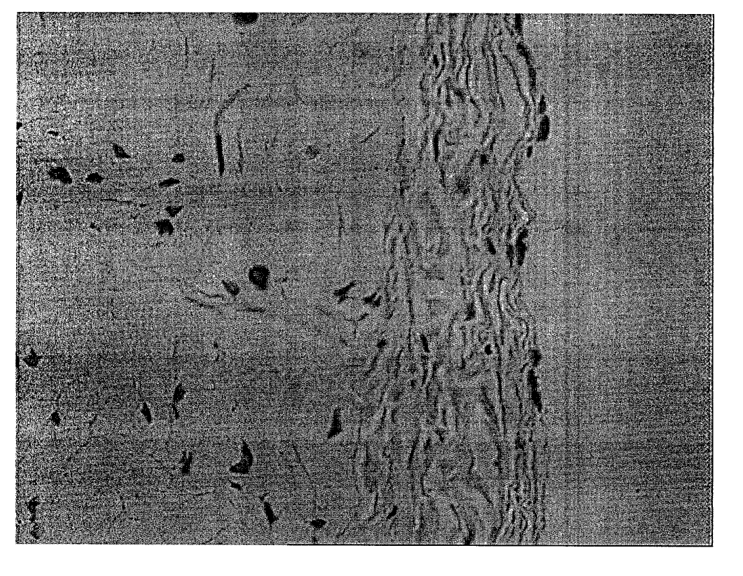

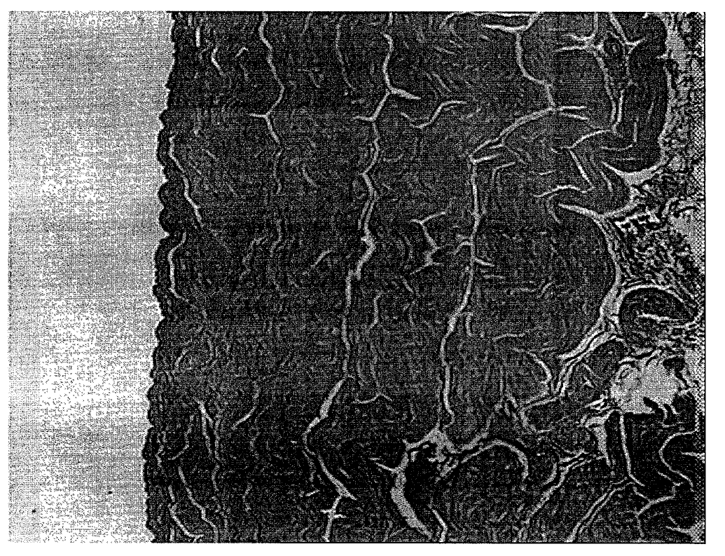

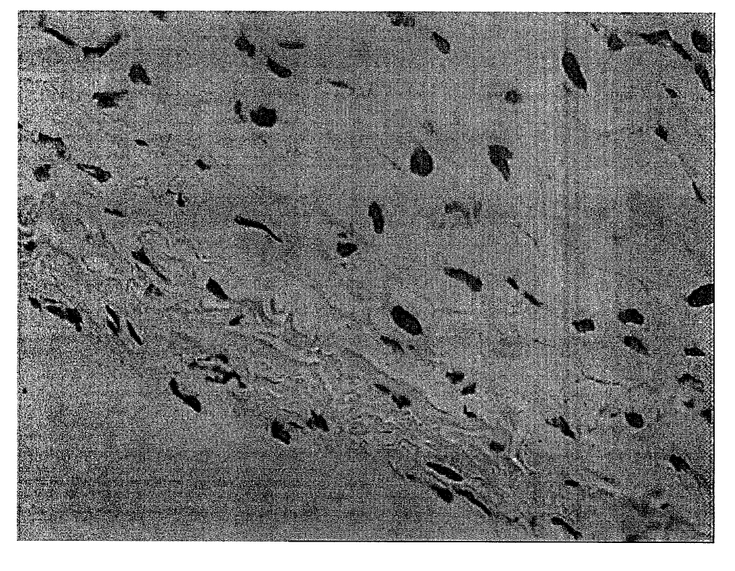

FIG. 4 is a photomicrograph the right upper pulmonary vein within 0.2 cm of the left atrial ostium of a canine subject. The medial layer contains a large number of atrial myocytes. After heating to 45 degrees C. for 20 minutes, no gross histological damage is seen (especially as compared to that demonstrated in FIG. 2). When the methods of the invention are applied as in FIG. 5, which is a photomicrograph of the TUNEL immunostain of the same vein as shown in FIG. 4, it is possible to demonstrate on positive endothelial and subendothelial cells, that gentle heat can produce apoptosis of atrial myocytes without necrosis, thrombosis, or inflammation. Similarly, in FIG. 6 can be seen a photomicrograph of the right lower pulmonary vein within 0.2 cm of the left atrial ostium of another canine subject. Again, the media contains a large number of atrial myocytes. And, again, after heating to 45 degrees for 20 minutes, no gross histological damage is seen. FIG. 7 likewise shows a photomicrograph of TUNEL stain on the section of same vein as in FIG. 6, demonstrating positive (brown) endothelial and subendothelial cells.

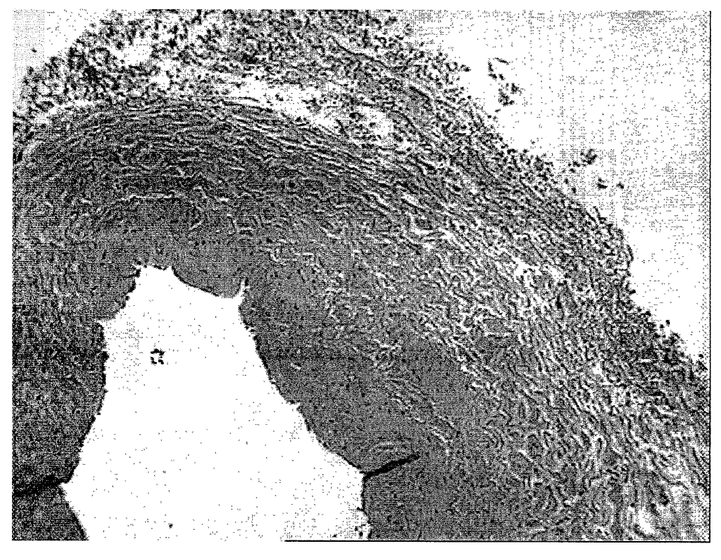

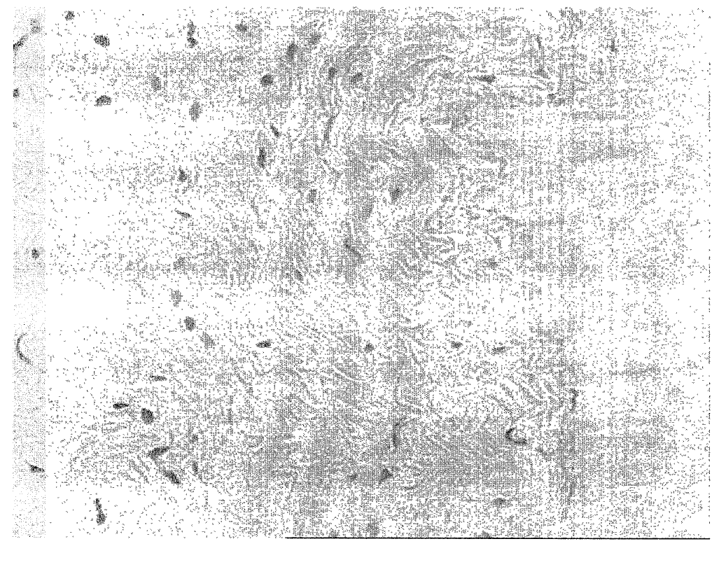

Negative controls were performed as recorded in FIGS. 8 and 9. These figures, respectively, are photomicrographs of the left upper pulmonary vein about 0.5 cm from the left atrial ostium in which no heating was performed, and in which using TUNEL immunostains, no apoptotic cells are seen. Thus, using the methods of the invention, it is possible to induce the cellular death of atrial myocytes which are typically the arrythmogenic foci of AF, without the serious collateral damage caused by prior art ablation techniques.

References

Benjamin E J, Levy D. Vaziri S M, et. al. Independent risk factors for atrial fibrillation in a population-based cohort: the Framingham Heart Study. JAMA 1994; 271:840-844.

Chen S, Hsieh M, Tai C, et. al. Circulation 1999; 100:1879-1886.

Feinberg W M, Blackshear J L, Laupacis A, et. al. Prevalence, age distribution, and gender of patents with atrial fibrillation. Arch Intern Med. 1995; 155:469-473.

Gold R L, Haffajee R I, Chros G, et. al. Amiodarone for refractory atrial fibrillation. Am J Cardiol. 1986; 57:124-127.

Gold R. Differentiation between cellular apoptosis and necrosis by combined use of in situ tailing translation techniques. Laboratory Investigation. 1994:71(2):219.

Haissaguerre M, Jais P, Shah D C, et. al. Spontaneous initiation of atrial fibrillation by ectopic beats originating in the pulmonary veins. N Engl J Med. 1998; 399:659-666.

Huang S K, Graham A R, Wharton K. Radiofrequency catheter inducing apoptosis of the left and right ventricles: Anatomic and electrophysiologic observations. Pacing Clin Electrophysiol. 1988; 11:449-459.

James T N. Normal and abnormal consequences of apoptosis in the human heart. Ann Rev Physiol 1998; 60:309-25.

Juul-Moller S, Edvardsson N, Rehnqvist-Ahlberg N. Sotalol versus quinidine for the maintenance of sinus rhythm after direct current cardioversion of atrial fibrillation. Circulation 1990; 82:1932-1939.

Prystowsky E N. Proarrhythmia during drug treatment of supraventricular tachycardia: paradoxical risk of sinus rhythm for sudden death. Am J Cariol. 1996; 78(8A): 35-41.

Wolf P A, Abbott R D, Kannel W B. Atrial fibrillation as an independent risk factor for stroke: the Framingham Study. Stroke 1991; 22:983-988.

Wolf P A, Benjamin E J, Belanger A J, et. al. Secular trends in the prevalence of atrial fibrillation: the Framingham Study. Am Heart J. 1996; 131:790-795.

All patents and publications mentioned in this specification are indicative of the level of skill of those of knowledge in the art to which the invention pertains. All patents and publications referred to in this application are incorporated herein by reference to the same extent as if each was specifically indicated as being incorporated by reference, and to the extent that they provide materials and methods not specifically shown.

While the preferred embodiment of the invention has been shown and described, modifications thereof can be made by one skilled in the art without departing from the spirit and teachings of the invention. The embodiments described herein are exemplary only, and are not limiting. Many variations and modifications of the methods and compositions of the invention disclosed herein are possible and are within the scope of the invention. Accordingly, the scope of protection is not limited by the description set out above, but is only limited by the claims which follow, that scope including all equivalents of the subject matter of the claims.

What is claimed is:

1. A method of treating atrial fibrillation comprising inducing apoptosis of an atrial myocyte.

2. The method of treating atrial fibrillation of claim 1 wherein said heating is conducted at a temperature in the range of about 38° C. to 48° C.

3. The method of treating atrial fibrillation of claim 1 wherein said heating is conducted at a temperature in the range of about 40° C. to 46° C.

4. The method of treating atrial fibrillation of claim 1 wherein said heating is conducted at a temperature in the range of about 42° C. to 44° C.

5. The method of treating atrial fibrillation of claim 1 wherein said heating is conducted over a time period range of about 5 to 60 minutes.

6. The method of treating atrial fibrillation of claim 1 wherein said heating is conducted over a time period range of about 5 to 30 minutes.

7. The method of treating atrial fibrillation of claim 1 wherein said heating is conducted over a time period range of about 5 to 15 minutes.

8. The method of treating atrial fibrillation of claim 1, said method further comprising detecting the presence of an arrhythmogenic focus comprising an atrial myocyte.

9. The method of treating atrial fibrillation of claim 8, wherein said detecting step comprises electrically detecting the presence of said atrial myocyte.

10. The method of treating atrial fibrillation of claim 8, wherein said detecting step comprises detecting the presence of said atrial myocyte using positron emission tomography.

11. The method of treating atrial fibrillation of claim 10, wherein said positron emission tomography tracks differential uptake of a radio-contrast agent by said atrial myocyte.

12. The method of treating atrial fibrillation of claim 11, wherein said radio-contrast agent is 18-fluorodeoxyglucose.

13. The method of treating atrial fibrillation of claim 8, wherein said detecting step comprises thermogenically detecting the presence of said atrial myocyte.

14. The method of treating atrial fibrillation of claim 1, said method further comprising monitoring apoptosis of said atrial myocyte.

15. The method of treating atrial fibrillation of claim 14, wherein said monitoring step comprises electrically monitoring for the presence of said atrial myocyte.

16. The method of treating atrial fibrillation of claim 14, wherein said monitoring step comprises detecting the presence of said atrial myocyte using positron emission tomography.

17. The method of treating atrial fibrillation of claim 16, wherein said positron emission tomography tracks differential uptake of a radio-contrast agent by said atrial myocyte.

18. The method of treating atrial fibrillation of claim 17, wherein said radio-contrast agent is 18-fluorodeoxyglucose.

19. The method of treating atrial fibrillation of claim 14, wherein said monitoring step comprises thermogenically detecting the presence of said atrial myocyte.

20. The method of treating atrial fibrillation of claim 14, wherein said thermogenic detection of the presence of said atrial myocyte further comprises using an infrared balloon catheter.

21. The method of treating atrial fibrillation of claim 1, additionally comprising applying at least one additional trigger of apoptosis to said atrial myocyte.

22. The method of treating atrial fibrillation of claim 21, wherein said additional trigger of apoptosis is selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte.

23. A method of treating atrial fibrillation comprising inducing apoptosis of an atrial myocyte by heating, wherein said heating is conducted at a temperature in the range of about 38° C. to 48° C. over a time period range of about 5 to 60 minutes.

24. A method of treating atrial fibrillation comprising inducing apoptosis of an atrial myocyte by heating, further comprising detecting said atrial myocyte, and heating said atrial myocyte at a temperature in the range of about 38° C. to 48° C. over a time period range of about 5 to 60 minutes.

25. A method of treating atrial fibrillation comprising inducing apoptosis of an atrial myocyte by heating, wherein said heating is conducted at a temperature in the range of about 38° C. to 48° C. over a time period range of about 5 to 60 minutes, and monitoring apoptosis of said atrial myocyte.

26. A method of treating atrial fibrillation comprising inducing apoptosis of an atrial myocyte by heating, further comprising detecting said atrial myocyte, heating said atrial myocyte at a temperature in the range of about 38° C. to 48° C. over a time period range of about 5 to 60 minutes, and monitoring apoptosis of said atrial myocyte.

27. A method of treating atrial fibrillation comprising inducing apoptosis of an atrial myocyte by heating, further comprising detecting said atrial myocyte, heating said atrial myocyte at a temperature in the range of about 38° C. to 48° C. over a time period range of about 5 to 60 minutes, and monitoring apoptosis of said atrial myocyte, and wherein at least one additional trigger of apoptosis is applied to said atrial myocyte, said trigger selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte.

28. An improved method of treating atrial fibrillation comprising: inducing apoptosis of an atrial myocyte by heating, further comprising detecting said atrial myocyte, heating said atrial myocyte at a temperature in the range of about 38° C. to 48° C. over a time period range of about 5 to 60 minutes, and monitoring apoptosis of said atrial myocyte, and wherein at least one additional trigger of apoptosis is applied to said atrial myocyte, said trigger selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte.

29. A method of eliminating an arrhythmogenic focus in a pulmonary vein comprising inducing apoptosis of an atrial myocyte by heating.

30. The method of eliminating an arrythmogenic focus in a pulmonary vein of claim 29 wherein said heating is conducted at a temperature in the range of about 38° C. to 48° C.

31. The method of eliminating an arrythmogenic focus in a pulmonary vein of claim 29 wherein said heating is conducted at a temperature in the range of about 40° C. to 46° C.

32. The method of eliminating an arrythmogenic focus in a pulmonary vein of claim 29 wherein said heating is conducted at a temperature in the range of about 42° C. to 44° C.

33. The method of eliminating an arrythmogenic focus in a pulmonary vein of claim 29 wherein said heating is conducted over a time period range of about 5 to 60 minutes.

34. The method of eliminating an arrythmogenic focus in a pulmonary vein of claim 29 wherein said heating is conducted over a time period range of about 5 to 30 minutes.

35. The method of eliminating an arrythmogenic focus in a pulmonary vein of claim 29 wherein said heating is conducted over a time period range of about 5 to 15 minutes.

36. The method of eliminating an arrhythmogenic focus in a pulmonary vein of claim 29, said method further comprising detecting the presence of an arrhythmogenic focus comprising atrial myocytes.

37. The method of eliminating an arrhythmogenic focus in a pulmonary vein of claim 36, wherein said detecting step comprises electrically detecting the presence of said atrial myocytes.

38. The method of eliminating an arrythmogenic focus in a pulmonary vein of claim 36, wherein said detecting step comprises detecting the presence of said atrial myocytes using positron emission tomography.

39. The method of eliminating an arrythmogenic focus in a pulmonary vein of claim 38, wherein said positron emission tomography tracks differential uptake of a radio-contrast agent by said atrial myocytes.

40. The method of eliminating an arrythmogenic focus in a pulmonary vein of claim 39, wherein said radio-contrast agent is 18-fluorodeoxyglucose.

41. The method of eliminating an arrythmogenic focus in a pulmonary vein of claim 36, wherein said detecting step comprises thermogenically detecting the presence of said atrial myocytes.

42. The method of eliminating an arrythmogenic focus in a pulmonary vein of claim 29, said method further comprising monitoring apoptosis of said atrial myocytes.

43. The method of eliminating an arrythmogenic focus in a pulmonary vein of claim 42, wherein said monitoring step comprises electrically detecting the presence of said atrial myocytes.

44. The method of eliminating an arrythmogenic focus in a pulmonary vein of claim 42, wherein said monitoring step comprises detecting the presence of said atrial myocytes using positron emission tomography.

45. The method of eliminating an arrythmogenic focus in a pulmonary vein of claim 44, wherein said positron emission tomography tracks differential uptake of a radio-contrast agent by said atrial myocytes.

46. The method of eliminating an arrythmogenic focus in a pulmonary vein of claim 45, wherein said radio-contrast agent is 18-fluorodeoxyglucose.

47. The method of eliminating an arrythmogenic focus in a pulmonary vein of claim 42, wherein said monitoring step comprises thermogenically detecting the presence of said atrial myocytes.

48. The method of eliminating an arrythmogenic focus in a pulmonary vein of claim 47, wherein said thermogenic detection of the presence of said atrial myocytes further comprises using an infrared ballon catheter.

49. The method of eliminating an arrythmogenic focus in a pulmonary vein of claim 29, wherein said additional trigger of apoptosis is selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte.

50. A method of eliminating an arrythmogenic focus in a pulmonary vein comprising inducing apoptosis of a group of atrial myocytes by heating, wherein said heating is conducted at a temperature in the range of about 38° C. to 48° C. over a time period range of about 5 to 60 minutes.

51. A method of eliminating an arrythmogenic focus in a pulmonary vein comprising inducing apoptosis of a group of atrial myocytes, further comprising detecting said atrial myocytes, and heating said atrial myocytes at a temperature in the range of about 38° C. to 48° C. over a time period range of about 5 to 60 minutes.

52. A method of eliminating an arrythmogenic focus in a pulmonary vein comprising inducing apoptosis of a group of atrial myocytes by heating, wherein said heating is conducted at a temperature in the range of about 38° C. to 48° C. over a time period range of about 5 to 60 minutes, and monitoring apoptosis of said atrial myocytes.

53. A method of eliminating an arrythmogenic focus in a pulmonary vein comprising inducing apoptosis of a group of atrial myocytes, further comprising detecting said atrial myocytes, heating said atrial myocytes at a temperature in the range of about 38° C. to 48° C. over a time period range of about 5 to 60 minutes, and monitoring apoptosis of said atrial myocytes.

54. A method of eliminating an arrhythmogenic foci in a pulmonary vein comprising inducing apoptosis of a group of atrial myocytes by heating.

55. A method of eliminating an arrythmogenic foci in a pulmonary vein comprising, detecting an atrial myocyte, heating said atrial myocyte at a temperature in the range of about 38° C. to 48° C. over a time period range of about 5 to 60 minutes in order to induce apoptosis in said atrial myocyte, applying at least one additional trigger of apoptosis to said atrial myocyte, said trigger selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte, and monitoring apoptosis of said atrial myocyte.

56. An improved method of eliminating an arrythmogenic foci in a pulmonary vein, comprising: detecting an atrial myocyte, heating said atrial myocyte at a temperature in the range of about 38° C. to 48° C. over a time period range of about 5 to 60 minutes in order to induce apoptosis in said atrial myocyte, applying at least one additional trigger of apoptosis to said atrial myocyte, said trigger selected from the group: application of tumor necrosis factor alpha to said atrial myocyte; pressure against said atrial myocyte; stretching said atrial myocyte; causing hypoxia in said atrial myocyte; causing hypoglycemia in said atrial myocyte; causing acidosis in said atrial myocyte; and application of oxidants to said atrial myocyte, and monitoring apoptosis of said atrial myocyte.

57. A device for eliminating an arrhythmogenic focus in a pulmonary vein comprising:
a heating element capable of maintaining a temperature in the range of about 38° C. to 48° C. over a time period range of about 5 to 60 minutes; and,
at least one detector capable of detecting the presence or absence of an arrhythmogenic focus comprising atrial myocytes.

58. The device of claim 57, wherein said detector electrically detects the presence of said atrial myocytes by their ectopic electronic emissions.

59. The device of claim 57, wherein said detector detects the presence of said atrial myocytes using positron emission tomography.

60. The device of claim 59, wherein said detector tracks differential uptake of a radio-contrast agent by said atrial myocytes using positron emission tomography.

61. The device of claim 57, wherein said detector is capable of thermogenically detecting the presence of said atrial myocytes.

62. The device of claim 57, wherein said detector is capable of monitoring apoptosis of said atrial myocytes.

63. The device of claim 57, wherein said detector is capable of both detecting the presence of atrial myocytes and of monitoring the removal of said atrial myocytes.
